(19) United States
(12) Patent Application Publication
(10) Pub. No.: US 2025/0344979 A1
(43) Pub. Date: Nov. 13, 2025

(54) PHYSIOLOGICAL SIGNAL MEASURING DEVICE AND PHYSIOLOGICAL SIGNAL MEASURING METHOD

(71) Applicant: Chung Yuan Christian University, Taoyuan City (TW)

(72) Inventors: Shao-Hung Lu, Taoyuan City (TW); En Chuang, Taoyuan City (TW); Shi-Yi Wu, Taoyuan City (TW); Mei-Fen Chen, Taoyuan City (TW); Wen-Chi Lin, Taoyuan City (TW); Cheng Lun Tsai, Taoyuan City (TW); Kang-Ping Lin, Taoyuan City (TW)

(73) Assignee: Chung Yuan Christian University, Taoyuan City (TW)

(21) Appl. No.: 18/973,094

(22) Filed: Dec. 8, 2024

(30) Foreign Application Priority Data
May 7, 2024 (TW) 113116829

(51) Int. Cl. A61B 5/307; A61B 5/00

(52) U.S. Cl. CPC A61B 5/307; A61B 5/7203

(57) ABSTRACT

A physiological signal measuring device and a physiological signal measuring method are provided. The physiological signal measuring device includes a first sensing electrode, a second sensing electrode, an amplifier, a calculator and a subtractor. The first sensing electrode has a first electrode impedance value. The first sensing electrode acquires the first electrode signal of a user. The second sensing electrode has a second electrode impedance value. The second sensing electrode acquires the second electrode signal of the user. The amplifier amplifies the second electrode signal to generate an amplified signal. The calculator generates a physiological signal according to the amplified signal and the first electrode signal. The subtractor subtracts the second electrode signal from the physiological signal to generate a biopotential differential signal.

### CROSS-REFERENCE TO RELATED APPLICATION

This application claims the priority benefit of Taiwan application serial no. 113116829, filed on May 7, 2024. The entirety of the above-mentioned patent application is hereby incorporated by reference herein and made a part of this specification.

### BACKGROUND

### Technical Field

The disclosure relates to a measuring device and a measuring method, and in particular to a physiological signal measuring device and a physiological signal measuring method.

### Description of Related Art

Generally speaking, physiological signal measuring devices use at least three sensing electrodes to acquire biopotential signals. The biopotential signal is, for example, an electrocardiogramaignal, an electroencephalography (EEG) signal, or an electromyography (EMG) signal. There is a power noise in biopotential signals. The power noise is a power line interference (PLI) signal. The physiological signal measuring device uses a first sensing electrode and a second sensing electrode to extract a common mode signal of the first sensing electrode and the second sensing electrode, and uses a third sensing electrode to reversely input the common mode signal back to the biological terminal to be measured to suppress the power noise. Therefore, the physiological signal measuring device can use the third sensing electrode to reduce the influence of the power noise on the biopotential signal.

In recent years, more and more studies have used physiological signal measuring devices with only first sensing electrodes and second sensing electrodes to measure biopotential signals. However, a physiological signal measuring device that only has the first sensing electrode and the second sensing electrode may not use other electrodes to reduce the interference from the power noise. Therefore, the physiological signal measuring device inverts the electrode signal from the second sensing electrode to generate an inverted signal, and adds the inverted signal to the electrode signal from the first sensing electrode to try to offset the power noise. It should be noted that an impedance of the first sensing electrode is different from an impedance of the second sensing electrode based on the actual difference in contact area between the electrode and the measured part and/or the actual difference in moisture content of the measured part. Therefore, the method does not eliminate the power noise in the biopotential signal. In other words, there is still power noise in the biopotential signal. Even if the biopotential signal is gain or amplified, the power noise is also gained or amplified. Therefore, a signal-to-noise ratio and a magnification of the biopotential signals are limited by the power noise.

### SUMMARY

The disclosure provides a physiological signal measuring device and a physiological signal measuring method that can generate a biopotential differential signal using a first electrode signal from a first sensing electrode and a second electrode signal from a second sensing electrode, and eliminate an influence of a power noise on the biopotential differential signal.

In an embodiment of the disclosure, a physiological signal measuring device includes a first sensing electrode, a second sensing electrode, an amplifier, a calculator, and a subtractor. The first sensing electrode has a first electrode impedance value. The first sensing electrode acquires a first electrode signal of a user. The first electrode signal includes a first biopotential signal and a first power noise component from a power noise. The second sensing electrode has a second electrode impedance value. The second sensing electrode acquires a second electrode signal of the user. The second electrode signal includes a second biopotential signal and a second power noise component from the power noise. The first electrode impedance value is (1+N) times the second electrode impedance value. The amplifier is electrically connected to the second sensing electrode. The amplifier amplifies the second electrode signal to generate an amplified signal. The calculator is electrically connected to the amplifier and the first sensing electrode. The calculator generates a physiological signal based on the amplified signal and the first electrode signal. The physiological signal is equal to a calculation result of the first electrode signal minus the N-fold amplified second electrode signal. The subtractor is electrically connected to the calculator and the second sensing electrode. The subtractor subtracts the second electrode signal from the physiological signal to generate a biopotential differential signal.

In an embodiment of the disclosure, the physiological signal measuring method includes: providing the first sensing electrode and the second sensing electrode, in which the first sensing electrode has the first electrode impedance value, in which the second sensing electrode has the second electrode impedance value, in which the first electrode impedance value is (1+N) times the second electrode impedance value; acquiring the first electrode signal of the user by the first sensing electrode and acquiring the second electrode signal of the user by the second sensing electrode, in which the first electrode signal includes the first biopotential signal and the first power noise component from the power noise, in which the second electrode signal includes the second biopotential signal and the second power noise component from the power noise; amplifying the second electrode signal to generate the amplified signal and generating the physiological signal based on the amplified signal and the first electrode signal, in which the physiological signal is equal to the calculation result of the first electrode signal minus the N-fold amplified second electrode signal; and subtracting the second electrode signal from the physiological signal to generate the biopotential differential signal.

Based on the above, the first electrode impedance value is (1+N) times the second electrode impedance value. The physiological signal is equal to the calculation result of the first electrode signal minus the N-fold amplified second electrode signal. In addition, the second electrode signal is subtracted from the physiological signal to generate the biopotential differential signal. It should be noted that when the second electrode signal is subtracted from the physiological signal, the influence of the power noise on the biopotential differential signal is eliminated. In this way, a signal-to-noise ratio of the biopotential differential signal is increased.

### BRIEF DESCRIPTION OF THE DRAWINGS

FIG. 1 is a schematic diagram of a physiological signal measuring device according to an embodiment of the disclosure.

FIG. 2 is an equivalent schematic diagram of the first sensing electrode and the second sensing electrode according to an embodiment of the disclosure.

FIG. 3 is a schematic diagram of a physiological signal measuring device according to an embodiment of the disclosure.

FIG. 4 is a schematic diagram of a physiological signal measuring device according to an embodiment of the disclosure.

FIG. 5 is a schematic diagram of a physiological signal measuring device according to an embodiment of the disclosure.

FIG. 6 is a schematic diagram of a physiological signal measuring device according to an embodiment of the disclosure.

FIG. 7 is a schematic diagram illustrating the use of first electrode signals and second electrode signals to obtain a magnification according to an embodiment of the disclosure.

FIG. 8 is a schematic diagram of a physiological signal measuring method according to an embodiment of the disclosure.

FIG. 9 is a waveform diagram of the biopotential signal.

FIG. 10 is a waveform diagram of a biopotential differential signal according to an embodiment of the disclosure.

FIG. 11 is a schematic diagram illustrating the use of a physiological signal measuring device according to an embodiment of the disclosure.

### DESCRIPTION OF THE EMBODIMENTS

Some embodiments of the disclosure will be described in detail below with reference to the accompanying drawings. Reference numerals quoted in the following description will be regarded as the same or similar components when the same reference numerals appear in different drawings. The embodiments are merely a part of the disclosure and do not disclose all possible implementations of the disclosure.

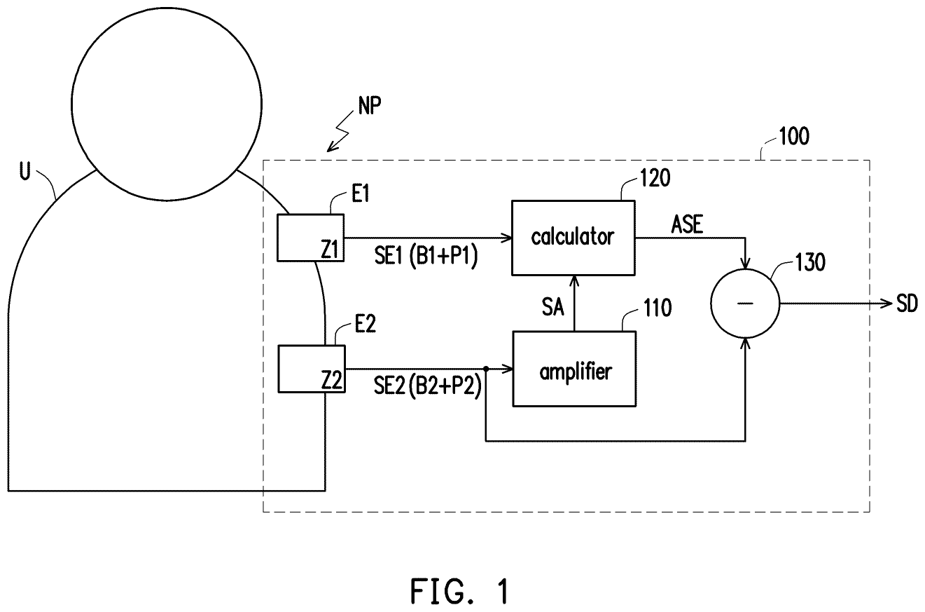

Referring to FIG. 1, FIG. is a schematic diagram of a physiological signal measuring device according to an embodiment of the disclosure. In this embodiment, a physiological signal measuring device 100 includes a first sensing electrode E1, a second sensing electrode E2, an amplifier 110, a calculator 120, and a subtractor 130. The first sensing electrode E1 has an electrode impedance value Z1. The first sensing electrode E1 acquires a first electrode signal SE1 of a user U. The first electrode signal SE1 includes a first biopotential signal B1 and a first power noise component P1 from a power noise NP.

The power noise NP is a power line interference (PLI) signal. Furthermore, the first power noise component P1 may be a noise component generated by the coupling of the power noise NP to the first electrode signal SE1.

In this embodiment, the second sensing electrode E2 has an electrode impedance value Z2. The second sensing electrode E2 acquires a second electrode signal SE2 of the user U. The second electrode signal SE2 includes a second biopotential signal B2 and a second power noise component P2 from the power noise NP. The second power noise component P2 may be a noise component generated by coupling of the power noise NP to the second sensing electrode E2.

For example, the first sensing electrode E1 is in contact with a first position of the user U to obtain the first electrode signal SE1. The second sensing electrode E2 is in contact with a second position of the user U to obtain the second electrode signal SE2.

In this embodiment, the electrode impedance value Z1 is (1+N) times the electrode impedance value Z2. N may be any real number. In other words, the electrode impedance value Z1 may be different from the electrode impedance value Z2. Generally, there is a first contact area between the first sensing electrode E1 and the first position. There is a second contact area between the second sensing electrode E2 and the second position. The first contact area may actually be different from the second contact area. Additionally, a moisture content at the first position may be different than a moisture content at the second position. The above situation may cause the electrode impedance value Z1 to be different from the electrode impedance value Z2.

In this embodiment, the amplifier 110 is electrically connected to the second sensing electrode E2. The amplifier 110 amplifies the second electrode signal SE2 to generate an amplified signal SA. The calculator 120 is electrically connected to the amplifier 110 and the first sensing electrode E1. The calculator 120 generates a physiological signal ASE based on the amplified signal SA and the first electrode signal SE1. In this embodiment, the physiological signal ASE is equal to a calculation result of the first electrode signal SE1 minus the N-fold amplified second electrode signal SE2. The subtractor 130 is electrically connected to the calculator 120 and the second sensing electrode E2. The subtractor 130 subtracts the second electrode signal SE2 from the physiological signal ASE to generate a biopotential differential signal SD.

It is worth mentioning here that when the second electrode signal SE2 is subtracted from the physiological signal ASE to generate the biopotential differential signal SD, the influence of the power noise NP on the biopotential differential signal SD is eliminated. In this way, a signal-to-noise ratio of the biopotential differential signal SD is increased.

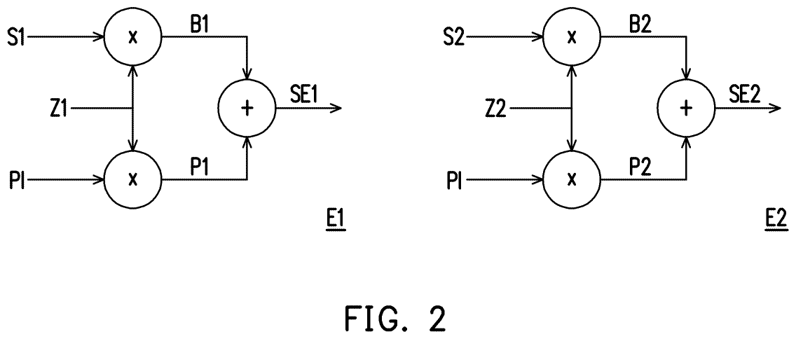

For specific explanation, referring to FIG. 1 and FIG. 2, FIG. 2 is an equivalent schematic diagram of the first sensing electrode and the second sensing electrode according to an embodiment of the disclosure. In this embodiment, the first sensing electrode E1 receives a biological signal S1 and a power coupling current value PI of the power noise NP. The biological signal S1 is a current value. The first sensing electrode E1 generates the first biopotential signal B1 based on the biological signal S1 and the electrode impedance value Z1 of the first sensing electrode E1. The first sensing electrode E1 generates the first power noise component P1 according to the power noise NP and the electrode impedance value Z1. The first sensing electrode E1 generates the first electrode signal SE1 according to the first biopotential signal B1 and the first power noise component P1. Furthermore, the first power noise component P1 is equal to a product of the power coupling current value PI of the power noise NP and the electrode impedance value Z1. The first biopotential signal B1 is equal to the product of the biological signal S1 and the electrode impedance value Z1. The first electrode signal SE1 is equal to the sum of the first power noise component P1 and the first biopotential signal B1. Therefore, the first electrode signal SE1 may be expressed by formula (1).

$$SE 1 = S 1 \times Z 1 + PI \times Z 1 \qquad formula ( 1 )$$

The second sensing electrode E2 receives a biological signal S2 and the power coupling current value PI of the power noise NP. The biological signal S2 is the current value. The second sensing electrode E2 generates the second biopotential signal B2 according to the biological signal S2 and the electrode impedance value Z2 of the second sensing electrode E2. The second sensing electrode E2 generates the second power noise component P2 according to the power noise NP and the electrode impedance value Z2. The second sensing electrode E2 generates the second electrode signal SE2 according to the second biopotential signal B2 and the second power noise component P2. Furthermore, the second power noise component P2 is equal to the product of the power coupling current value PI of the power noise NP and the electrode impedance value Z2. The second biopotential signal B2 is equal to the product of the biological signal S2 and the electrode impedance value Z2. The second electrode signal SE2 is equal to the sum of the second power noise component P2 and the second biopotential signal B2. Therefore, the second electrode signal SE2 may be expressed by formula (2).

$$SE 2 = S 2 \times Z 2 + PI \times Z 2 \qquad formula ( 2 )$$

The electrode impedance value Z1 is (1+N) times the electrode impedance value Z2. Therefore, the formula (1) is rewritten as formula (3).

$$SE 1 = S 1 \times ( 1 + N ) \times Z 2 + PI \times ( 1 + N ) \times Z 2 \qquad formula ( 3 )$$

In this embodiment, the amplifier 110 may be implemented by an inverting amplifier. The calculator 120 may be implemented by an adder. The amplifier 110 amplifies the second electrode signal SE2 by (−N) times to generate the amplified signal SA. The calculator 120 adds the amplified signal SA to the first electrode signal SE1 to generate the physiological signal ASE. The physiological signal ASE may be expressed by formula (4).

$$ASE = SE 1 - N \times SE 2 = [ S 1 \times ( 1 + N ) \times Z 2 + PI \times ( 1 + N ) \times Z 2 ] - N \times ( S 2 \times Z 2 + PI \times Z 2 ) \qquad formula ( 4 )$$

The formula (4) is simplified to formula (5)

$$ASE = S 1 \times ( 1 + N ) \times Z 2 + PI \times Z 2 - N \times S 2 \times Z 2 \qquad formula ( 5 )$$

Next, the subtractor 130 subtracts the second electrode signal SE2 from the physiological signal ASE to generate the biopotential differential signal SD. Therefore, the biopotential differential signal SD may be expressed by formula (6).

$$SD = ASE - SE 2 = ( 1 + N ) \times Z 2 \times ( S 1 - S 2 ) \qquad formula ( 6 )$$

Further, the formula (6) may be rewritten as formula (7).

$$SD = Z 1 \times ( S 1 - S 2 ) \qquad formula ( 7 )$$

It should be noted that in the formulas (6) and (7), when the biopotential differential signal SD is generated, the power coupling current value PI has been eliminated. Therefore, the biopotential differential signal SD is related to the electrode impedance value Z1 and a differential result of the biological signals S1 and S2.

In some embodiments, the amplifier 110 amplifies the second electrode signal SE2 by (N) times to generate the amplified signal SA. The calculator 120 may be implemented by a subtractor. The calculator 120 subtracts the amplified signal SA from the first electrode signal SE1 to generate the physiological signal ASE. The physiological signal ASE is also expressed by the formula (4).

In this embodiment, the physiological signal measuring device 100 may be, for example, an electrocardiogrameasuring device, an electroencephalography (EEG) measuring device, or an electromyography (EMG) measuring device. However, the disclosure is not limited thereto. Therefore, the biopotential differential signal SD may be an electrocardiogram signal, an electroencephalogram signal, or an electromyogram signal, but the disclosure is not limited thereto.

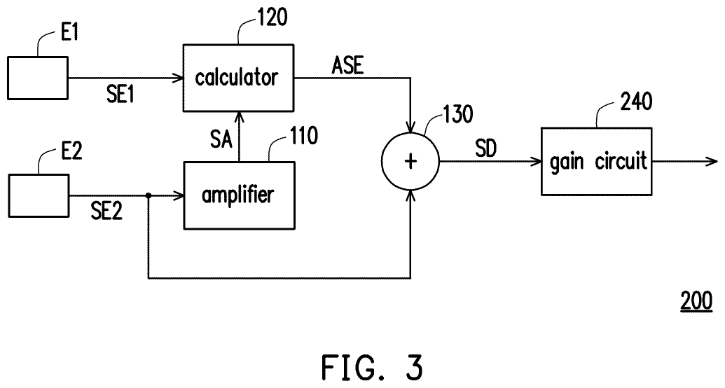

Referring to FIG. 3, FIG. 3 is a schematic diagram of a physiological signal measuring device according to an embodiment of the disclosure. In this embodiment, a physiological signal measuring device 200 includes the first sensing electrode E1, the second sensing electrode E2, the amplifier 110, the calculator 120, the subtractor 130, and a gain circuit 240. The implementation details of the first sensing electrode E1, the second sensing electrode E2, the amplifier 110, the calculator 120, and the subtractor 130 have been clearly explained in the embodiments of FIG. 1 and FIG. 2 and are not repeated herein.

In this embodiment, the gain circuit 240 is electrically connected to the subtractor 130. The gain circuit 240 gains the biopotential differential signal SD. For example, the gain circuit 240 may amplify a waveform of the biopotential differential signal SD by (M) times to generate a gain biological signal SD′.

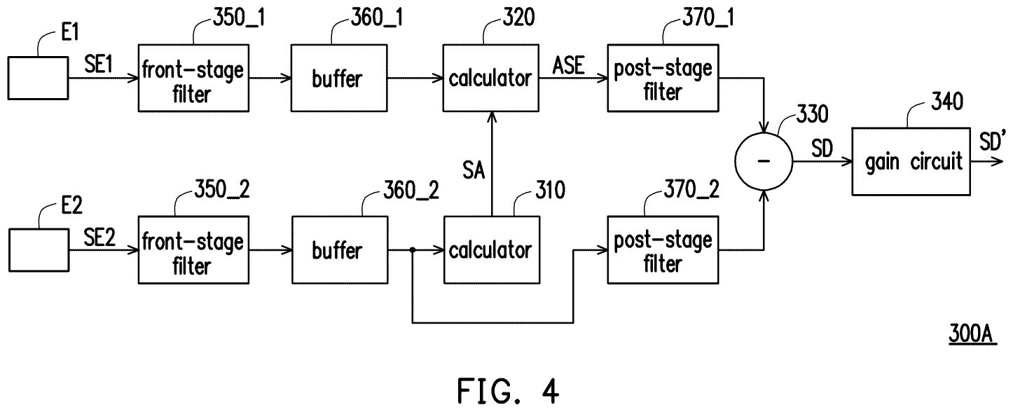

Referring to FIG. 4, FIG. 4 is a schematic diagram of a physiological signal measuring device according to an embodiment of the disclosure. In this embodiment, a physiological signal measuring device 300 includes the first sensing electrode E1, the second sensing electrode E2, an amplifier 310, a calculator 320, a subtractor 330, a gain circuit 340, front-stage filters 350_1 and 350_2, and buffers 360_1 and 360_2, and post-stage filters 370_1 and 370_2. The implementation details of the first sensing electrode E1 and the second sensing electrode E2 have been clearly explained in the embodiments of FIG. 1 and FIG. 2 and are not repeated herein.

In this embodiment, the front-stage filter 350_1 is electrically connected to the first sensing electrode E1. The front-stage filter 350_1 filters out a noise (e.g., a high-frequency noise) received when the first electrode signal SE1 is transmitted. The front-stage filter 350_2 is electrically connected to the second sensing electrode E2. The front-stage filter 350_2 filters out the noise (e.g., the high-frequency noise) received when the second electrode signal SE2 is transmitted.

In this embodiment, the buffer 360_1 is electrically connected to the front-stage filter 350_1. Generally speaking, the filtering operation of the front-stage filter 350_1 may reduce an intensity of the first electrode signal SE1. The buffer 360_1 may compensate the intensity of the first electrode signal SE1.

The buffer 360_2 is electrically connected to the front-stage filter 350_2 and the amplifier 310. Generally speaking, the filtering operation of the front-stage filter 350_2 may reduce the intensity of the second electrode signal SE2. The buffer 360_2 may compensate the intensity of the second electrode signal SE2.

In this embodiment, the buffers 360_1 and 360_2 are any type of voltage adjustment circuit, voltage dividing circuit, voltage compensation circuit, or impedance matching circuit respectively.

In this embodiment, the amplifier 310 receives the second electrode signal SE2 from the buffer 360_2. The amplifier 310 amplifies the second electrode signal SE2 by (−N) times, for example, to generate the amplified signal SA. The calculator 320 is electrically connected to the amplifier 310 and the buffer 360_1. The calculator 320 adds the amplified signal SA to the first electrode signal SE1 from the buffer 360_1 to generate the physiological signal ASE.

In this embodiment, the post-stage filter 370_1 is electrically connected to the buffer 360_1 and the subtractor 330. The post-stage filter 370_1 filters out the noise received when the physiological signal ASE is transmitted. The post-stage filter 370_2 is electrically connected to the buffer 360_2 and the subtractor 330. The post-stage filter 370_2 filters out the noise (e.g., high-frequency noise) received when the second electrode signal SE2 is transmitted. The subtractor 130 subtracts the second electrode signal SE2 from the subsequent filter 370_2 from the physiological signal ASE from the subsequent filter 370_1 to generate the biopotential differential signal SD.

In some embodiments, the gain circuit 340 may be omitted. In some embodiments, the front-stage filters 350_1 and 350_2 may be omitted. In some embodiments, the buffers 360_1 and 360_2 may be omitted. In some embodiments, the post-stage filters 370_1 and 370_2 may be omitted.

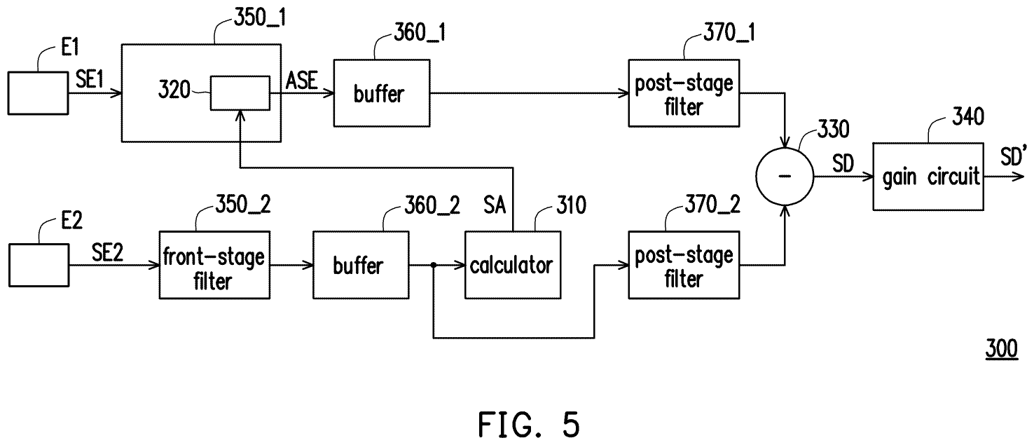

Referring to FIG. 5, FIG. 5 is a schematic diagram of a physiological signal measuring device according to an embodiment of the disclosure. In this embodiment, a physiological signal measuring device 300A includes the first sensing electrode E1, the second sensing electrode E2, the amplifier 310, the calculator 320, the subtractor 330, the gain circuit 340, the front-stage filters 350_1 and 350_2, the buffers 360_1 and 360_2, and the post-stage filters 370_1 and 370_2. Different from the physiological signal measuring device 300 in FIG. 4, the calculator 320 of the physiological signal measuring device 300A is provided in the front-stage filter 350_1. Therefore, the buffer 360_1 is used to compensate the intensity of the physiological signal ASE.

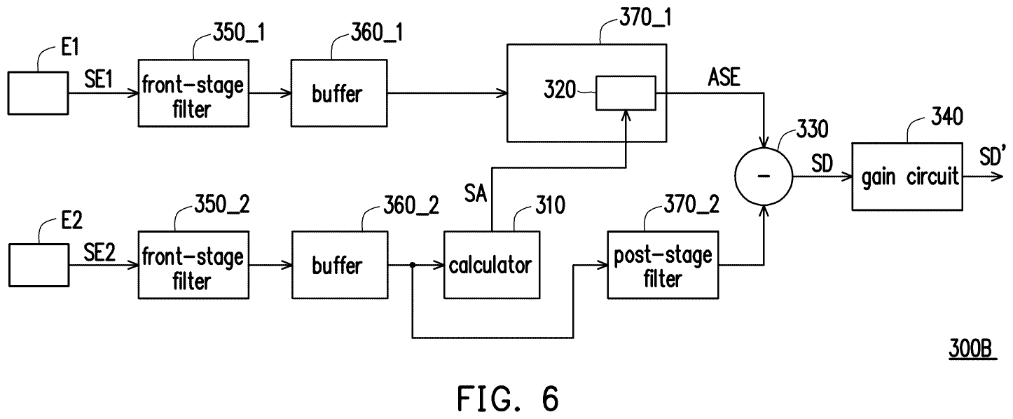

Referring to FIG. 6, FIG. 6 is a schematic diagram of a physiological signal measuring device according to an embodiment of the disclosure. In this embodiment, a physiological signal measuring device 300B includes the first sensing electrode E1, the second sensing electrode E2, the amplifier 310, the calculator 320, the subtractor 330, the gain circuit 340, the front-stage filters 350_1 and 350_2, the buffers 360_1 and 360_2, and the post-stage filters 370_1 and 370_2. Different from the physiological signal measuring device 300 in FIG. 4, the calculator 320 of the physiological signal measurement device 300B is provided in the post-stage filter 370_1. In this embodiment, the post-stage filter 370_1 may filter out the noise received when at least one of the first electrode signal SE1 and the physiological signal ASE is transmitted.

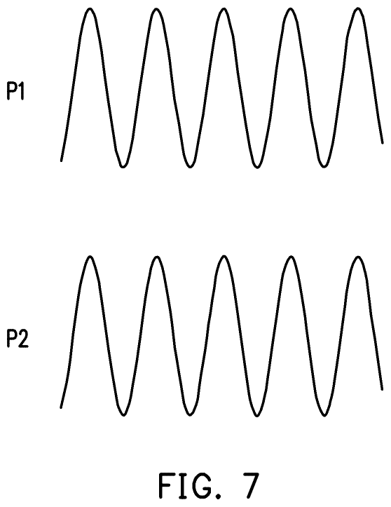

Next, an embodiment of determining a magnification ratio between the electrode impedance value Z1 of the first sensing electrode E1 and the electrode impedance value Z2 of the second sensing electrode E2 are described with reference to FIGS. 1 and 7. Refer to FIG. 1 and FIG. 7, FIG. 7 is a schematic diagram illustrating the use of first electrode signals and second electrode signals to obtain a magnification according to an embodiment of the disclosure. In a stage of determining the magnification, the first power noise component P1 of the first electrode signal SE1 is sampled. The second power noise component P2 of the second electrode signal SE2 is sampled. At this time, the frequencies of the first power noise component P1 and the second power noise component P2 are approximately equal to the frequency of the power noise NP (e.g., 50 Hz or 60 Hz).

In this embodiment, the first electrode signal SE1 and the second electrode signal SE2 are compared. When an intensity of the first power noise component P1 is equal to an intensity of the second power noise component P2, it indicates that the electrode impedance value Z1 of the first sensing electrode E1 is the same as the electrode impedance value Z2 of the second sensing electrode E2. That is, (N) is equal to 0.

For example, when the intensity of the first power noise component P1 is greater than the intensity of the second power noise component P2, it indicates that the electrode impedance value Z1 of the first sensing electrode E1 is greater than the electrode impedance value Z2 of the second sensing electrode E2. That is, (N) is greater than 0. Therefore, the waveform of the second power noise component P2 is amplified until being equal to the waveform of the first power noise component P1. Therefore, the value of (1+N) may be acquired. In this embodiment, the operations may be performed, for example, by the physiological signal measuring device 100. For example, the physiological signal measuring device 100 performs the operations in a calibration stage to acquire the value of (1+N).

In some cases, when the intensity of the first power noise component P1 is smaller than the intensity of the second power noise component P2, the waveform of the first power noise component P1 is amplified until being equal to the waveform of the second power noise component P2. Therefore, the value of (1+N) may also be acquired.

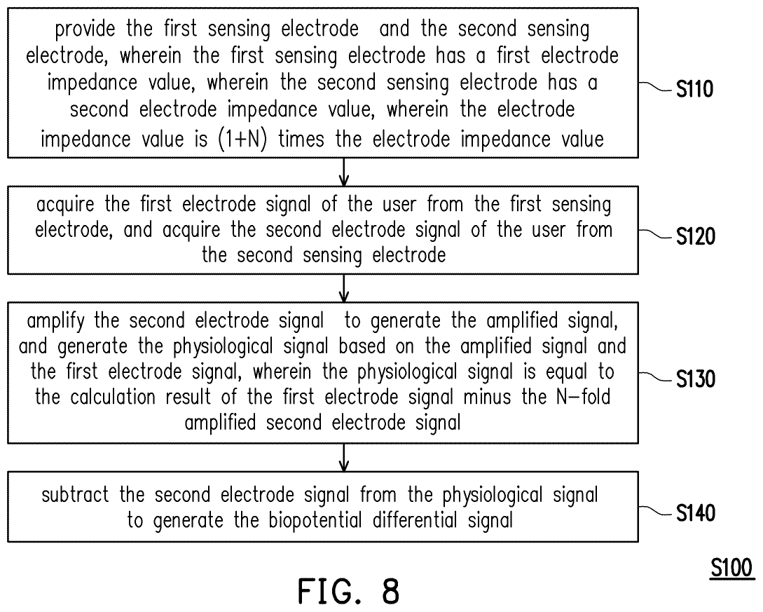

Referring to FIG. 1 and FIG. 8, FIG. 8 is a schematic diagram of a physiological signal measuring method according to an embodiment of the disclosure. In this embodiment, a physiological signal measuring method S100 includes steps S110 to S140. In step S110, the first sensing electrode E1 and the second sensing electrode E2 are provided. Therefore, the physiological signal measuring method S100 is suitable for the physiological signal measuring device 100. In this embodiment, the first sensing electrode E1 has an electrode impedance value Z1 (i.e., a first electrode impedance value). The second sensing electrode E2 has an electrode impedance value Z2 (i.e., a second electrode impedance value). The electrode impedance value Z1 is (1+N) times the electrode impedance value Z2. In step $120, the first sensing electrode E1 obtains the first electrode signal SE1 of the user U. The second sensing electrode E2 obtains the second electrode signal SE2 of the user U.

In step S130, the second electrode signal SE2 is amplified to generate the amplified signal SA. In addition, in step S130, the physiological signal ASE is generated based on the amplified signal SA and the first electrode signal SE1. In this embodiment, the physiological signal ASE is equal to the calculation result of the first electrode signal SE minus the N-fold amplified second electrode signal SE2. In step S140, the second electrode signal SE2 is subtracted from the physiological signal ASE to generate the biopotential differential signal SD.

The implementation details of the steps S110 to S140 have been clearly explained in the embodiments of FIG. 1 and FIG. 2 and are not repeated herein.

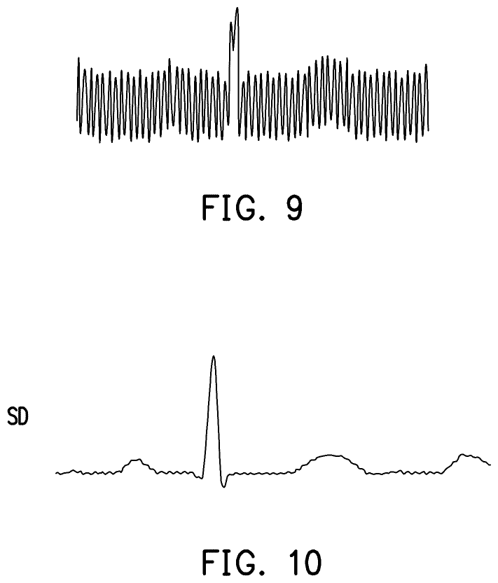

Referring to FIG. 1, FIG. 2, FIG. 9, and FIG. 10, FIG. 9 is a waveform diagram of the biopotential signal. FIG. 10 is a waveform diagram of a biopotential differential signal according to an embodiment of the disclosure. FIG. 9 shows the biopotential signal affected by the power noise NP. The biopotential signals have voltage fluctuations at a fixed frequency. The voltage fluctuation is caused by a coupling effect of the power noise NP. The voltage fluctuations significantly reduces the signal-to-noise ratio of the biopotential signal.

In this embodiment, the physiological signal measuring device 100 has eliminated the influence of the power noise NP on the biopotential differential signal SD. Therefore, the biopotential differential signal SD is related to the electrode impedance value Z1 and the differential results of the biological signals S1 and S2. It may be seen from this that the biopotential differential signal SD generated by the physiological signal measuring device 100 has a very high signal-to-noise ratio.

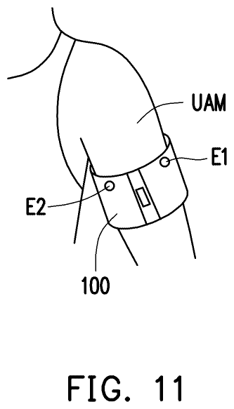

Referring to FIG. 1 and FIG. 11, FIG. 11 is a schematic diagram showing the use of a physiological signal measuring device according to an embodiment of the disclosure. In this embodiment, the physiological signal measuring device 100 is, for example, a single-arm measuring device (but the disclosure is not limited thereto). The first sensing electrode E1, the second sensing electrode E2, the amplifier 110, the calculator 120, and the subtractor 130 of the physiological signal measuring device 100 are arranged in a fixed unit (but the disclosure is not limited thereto). The fixed unit may fix the physiological signal measuring device 100 to an upper arm UAM of the user U (such as a left upper arm, but the disclosure is not limited thereto). When the fixed unit fixes the physiological signal measuring device 100 to the upper arm UAM of the user U, the first sensing electrode E1 is in contact with a first position of the upper arm UAM to acquire the first electrode signal SE1, and the second sensing electrode E2 is in contact with a second position of the upper arm part UAM to acquire the second electrode signal SE2.

In this embodiment, the fixed unit may be implemented by an elastic arm band with a fixing function (but the disclosure is not limited thereto).

In some embodiments, the first sensing electrode E1 and the second sensing electrode E2 of the physiological signal measuring device 100 are disposed on the fixed unit. The amplifier 110, the calculator 120, and the subtractor 130 are arranged outside the fixed unit.

To sum up, the first electrode impedance value is (1+N) times the second electrode impedance value. The physiological signal is equal to the calculation result of the first electrode signal minus the N-fold amplified second electrode signal. In addition, the second electrode signal is subtracted from the physiological signal to generate the biopotential differential signal. When the second electrode signal is subtracted from the physiological signal, the influence of power noise on the biopotential differential signal is eliminated. In this way, the disclosure can increase the signal-to-noise ratio of the biopotential differential signal.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A physiological signal measuring device, comprising:
a first sensing electrode, having a first electrode impedance value and configured to acquire a first electrode signal of a user, wherein the first electrode signal comprises a first biopotential signal and a first power noise component from a power noise;
a second sensing electrode, having a second electrode impedance value and configured to acquire a second electrode signal of the user, wherein the second electrode signal comprises a second biopotential signal and a second power noise component from the power noise, wherein the first electrode impedance value is (1+N) times the second electrode impedance value;
an amplifier, electrically connected to the second sensing electrode and configured to amplify the second electrode signal to generate an amplified signal;
an calculator, electrically connected to the amplifier and the first sensing electrode and configured to generate a physiological signal based on the amplified signal and the first electrode signal, wherein the physiological signal is equal to a calculation result of the first electrode signal minus the N-fold amplified second electrode signal; and
a subtractor, electrically connected to the calculator and the second sensing electrode and configured to subtract the second electrode signal from the physiological signal to generate a biopotential differential signal.

2. The physiological signal measuring device according to claim 1, wherein:
the first power noise component is equal to a product of a power coupling current value of the power noise and the first electrode impedance value, and
the second power noise component is equal to a product of the power coupling current value and the second electrode impedance value.

3. The physiological signal measuring device according to claim 1, wherein:
the amplifier amplifies the second electrode signal (−N) times to generate the amplified signal, and
the calculator adds the amplified signal to the first electrode signal to generate the physiological signal.

4. The physiological signal measuring device according to claim 1, wherein
the amplifier amplifies the second electrode signal (N) times to generate the amplified signal, and
the calculator subtracts the amplified signal from the first electrode signal to generate the physiological signal.

5. The physiological signal measuring device according to claim 1, further comprising:
a first front-stage filter, electrically connected to the first sensing electrode and configured to filter a noise from the first electrode signal; and
a second front-stage filter, electrically connected to the second sensing electrode and configured to filter a noise of the second electrode signal.

6. The physiological signal measuring device according to claim 5, wherein the calculator is disposed in the first front-stage filter.

7. The physiological signal measuring device according to claim 5, further comprising:
a first buffer, electrically connected to the first front-stage filter and configured to compensate an intensity of one of the first electrode signal and the physiological signal; and
a second buffer, electrically connected to the second front-stage filter and configured to compensate an intensity of the second electrode signal.

8. The physiological signal measurement device according to claim 7, further comprising:
a first post-stage filter, electrically connected to the first buffer and configured to filter out a noise received when at least one of the first electrode signal and the physiological signal is transmitted; and
a second post-stage filter, electrically connected to the second buffer and configured to filter out a noise received when the second electrode signal is transmitted.

9. The physiological signal measuring device according to claim 8, wherein the calculator is disposed in the first post-stage filter.

10. The physiological signal measuring device according to claim 1, further comprising:
a gain circuit, electrically connected to the subtractor and configured to gain the biopotential differential signal.

11. A physiological signal measuring method, comprising:
providing a first sensing electrode and a second sensing electrode, wherein the first sensing electrode has a first electrode impedance value, wherein the second sensing electrode has a second electrode impedance value, wherein the first electrode impedance value is (1+N) times the second electrode impedance value;
acquiring a first electrode signal of a user by the first sensing electrode and acquiring a second electrode signal of the user by the second sensing electrode, wherein the first electrode signal comprises a first biopotential signal and a first power noise component from a power noise, wherein the second electrode signal comprises a second biopotential signal and a second power noise component from the power noise;
amplifying the second electrode signal to generate an amplified signal and generating a physiological signal based on the amplified signal and the first electrode signal, wherein the physiological signal is equal to a calculation result of the first electrode signal minus the N-fold amplified second electrode signal; and
subtracting the second electrode signal from the physiological signal to generate a biopotential differential signal.

12. The physiological signal measuring method according to claim 11, wherein:
the first power noise component is equal to a product of a power coupling current value of the power noise and the first electrode impedance value, and
the second power noise component is equal to a product of the power coupling current value and the second electrode impedance value.

13. The physiological signal measuring method according to claim 11, wherein amplifying the second electrode signal to generate the amplified signal and generating the physiological signal based on the amplified signal and the first electrode signal comprises:
amplifying the second electrode signal (−N) times to generate the amplified signal; and
adding the amplified signal to the first electrode signal to generate the physiological signal.

14. The physiological signal measuring method according to claim 11, wherein amplifying the second electrode signal to generate the amplified signal and generating the physiological signal based on the amplified signal and the first electrode signal comprises:
amplifying the second electrode signal (N) times to generate the amplified signal; and
subtracting the amplified signal from the first electrode signal to generate the physiological signal.
